(19) United States
(12) Patent Application Publication
(10) Pub. No.: US 2015/0173804 A1
(43) Pub. Date: Jun. 25, 2015

(54) SPINE SYSTEM AND KIT

(71) Applicant: CARBOFIX IN ORTHOPEDICS LLC, New York, NY (US)

(72) Inventor: Reuven Gepstein, Kfar Saba (IL)

(73) Assignee: CARBOFIX IN ORTHOPEDICS LLC a corporation

(21) Appl. No.: 14/416,307

(22) PCT Filed: Jul. 7, 2013
(86) PCT No.: PCT/IL2013/050581

§ 371 Date: Jan. 22, 2015

Related U.S. Application Data
(60) Provisional application No. 61/674,931, filed on Jul. 24, 2012.

(51) Int. Cl. A61B 17/70

(52) U.S. Cl. CPC A61B 17/7037; A61B 17/7002; A61B 2017/564

(57) ABSTRACT

A spine system may include a rod, one or a plurality of screws and one or a plurality of screw connectors for connecting said one or a plurality of screws to the rod, all of which are solely or mostly made of a composite material.

### BACKGROUND

Spinal fusion is a common surgery for treatment of spinal pathologies. Typically, metal implants are used for this purpose, e.g., intra-pedicular screws, hooks and rods. However, even after major surgery, surgery failures are reported in about 20% to 30% of patients.

The cause of this failure is unknown. Current imaging techniques are not sufficient to reveal the cause of such failure. Computerized tomography (CT) imaging may not always give good visualization of the areas of interest due to masking effects of metal implants located near the pathology (nerves, discs, joints, etc.). Using Magnetic Resonance Imaging (MRI) may be inappropriate because of the existence of artifacts around the imaged metal implants in the patient's body near the pathology. Moreover, follow up of the surgery for evaluation of, for example, tumor expansion, deterioration in oncology cases, or evaluation of bone fusion is also impeded by metallic artifacts which are present in various imaging techniques. As a result, a spinal surgeon may be compelled to perform second and third operations in order to remove the metal implants, obtain a better image of the pathology so as to determine causes of the failure and decide on appropriate treatment.

A possible solution to this problem is to use implants made of a composite material instead of metallic implants. Composite material implants, such as Carbon fibers reinforced PolyEtherEtherKetone (PEEK) implants do not interfere with imaging techniques and allow clear view which is required for evaluation of post-operation conditions. Moreover, composite materials have better elasticity than metal implants, and can adapt to the individual condition and pathology of each patient. Due to the similarity of the elasticity of composite materials to the elasticity of bone, stress shielding phenomena is less likely to occur, which may lead to fewer stress fractures of implants and bone and fewer loosening of screws. Hence, in some cases, a bone graft may not be necessary in dynamic rod usage, such as in spinal fixation mode.

Composite carbon polymer materials are very strong (for example, carbon fiber is about five times stronger than titanium alloy in withstanding tension), and are commonly used in the aircraft industry. Furthermore, these materials have also been used in spine surgery (e.g. carbon PEEK cages). Recently it was suggested to make intra-pedicular screws, hooks and reinforced rods for spinal fusion of composite materials (WO 2011/111048).

### SUMMARY OF THE INVENTION

There is thus provided, according to embodiments of the present invention, a spine system. The system may include a rod, one or a plurality of screws; and one or a plurality of screw connectors for connecting said one or a plurality of screws to the rod, all of which are solely or mostly made of a composite material.

Furthermore, according to some embodiments of the present invention, the composite material may be selected from the group of composite materials that consist of: carbon polymer composite materials, PEEK, PEEK reinforced with about 60% by volume of carbon fibers, 60%-65% by volume of carbon fibers embedded in PEEK.

Furthermore, according to some embodiments of the present invention, the rod may include two opposite ends and includes a bulbous head on at least one of the two opposite ends.

Furthermore, according to some embodiments of the present invention, the rod may include a bulbous head on both of the two opposite ends.

Furthermore, according to some embodiments of the present invention, each of the screws may include a duct for mounting the screw over a guide-wire to guide that screw to a target implantation location.

Furthermore, according to some embodiments of the present invention, each of the screw connectors may be integral to a screw of said one or a plurality of screws.

Furthermore, according to some embodiments of the present invention, each of the screw connectors may be integral to the rod.

Furthermore, according to some embodiments of the present invention, each of the screw connectors may include a body with a bore to allow a portion of the rod to be inserted through the bore, and a screw top to engage with that screw connector so as to fasten that screw connector to the rod.

Furthermore, according to some embodiments of the present invention, the system may further include fastener beads, over which the screw connectors may be mounted and fastened.

Furthermore, according to some embodiments of the present invention, each of the fastener beads may include a spherical body, a bore through the body and a slit across to facilitate flexibility of that bead when mounting over the rod.

Furthermore, according to some embodiments of the present invention, the system may further include a heating device for heating the rod so as to allow bending it to a desired shape.

Furthermore, according to some embodiments of the present invention, the heating device may include a clamp for clamping a section of the rod to be heated.

Furthermore, according to some embodiments of the present invention, there is provided a spine system kit. The kit may include a plurality of rods, a plurality of screws and a plurality of screw connectors each for connecting a screw of said plurality of screws to the rod, all of which are solely or mostly made of a composite material.

Furthermore, according to some embodiments of the present invention, the kit may further include a plurality of fastener beads, over which each of said plurality of screw connectors is to be mounted and fastened.

Furthermore, according to some embodiments of the present invention,

### BRIEF DESCRIPTION OF THE DRAWINGS

The subject matter regarded as the invention is distinctly claimed in the concluding portion of the specification. The invention, however, both as to organization and method of operation, together with objects, features and advantages thereof, may best be understood by reference to the following detailed description when read with the accompanied drawings in which:

FIG. 1A is an angled view of a screw, in particular suitable for serving as an intra-pedicular screw, in accordance with an embodiment of the present invention;

FIG. 1B is a cross-sectional view of the screw depicted in FIG. 1A;

FIG. 2A illustrates a spine system fixation assembly with two end screws, in accordance with an embodiment of the present invention;

FIG. 2B illustrates a fixation rod of the fixation assembly shown in FIG. 2A;

FIG. 3A illustrates a spine system fixation assembly with one end screw and two intermediate screws, in accordance with an embodiment of the present invention;

FIG. 3B is a cross-sectional view of the spine system fixation assembly shown in FIG. 3A;

FIG. 3C illustrates a connecting rod of the spine system fixation assembly shown in FIG. 3A;

FIG. 3D illustrates a fastener bead of the spine system fixation assembly shown in FIG. 3A;

FIG. 3E illustrates the connecting rod of the spine system fixation assembly shown in FIG. 3A with several fastening beads;

FIG. 4A illustrates a connecting rod for use in a spine system fixation assembly, in accordance with embodiments of the present invention;

FIG. 4B illustrates a spine system fixation assembly, with two end screws, in accordance with embodiments of the present invention;

FIG. 4C is a cross-sectional view of the spine system fixation assembly shown in FIG. 4B;

FIG. 5A is an angled view of a screw, in particular suitable for serving as an intra-pedicular screw, in accordance with other embodiments of the present invention;

FIG. 5B is a lateral view of the screw shown in FIG. 5A;

FIG. 5C is a cross-sectional view of the screw shown in FIG. 5A;

FIG. 6 is an angles view of a screw connector, for connecting a screw to a rod of a spine system fixation assembly, in accordance with an embodiment of the present invention;

FIG. 7A illustrates a heating device for heating a rod of a spine system fixation assembly, to allow reshaping the rod, in accordance with an embodiment of the present invention;

FIG. 7B is a cross-sectional view of the functional end of the heating device shown in FIG. 7A; and

FIG. 7C is a schematic illustration of a heating device for heating a rod of a spine system fixation assembly, to allow reshaping the rod, in accordance with an embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those of ordinary skill in the art that the present invention may be practiced without these specific details. In other instances, well-known methods, procedures, components and circuits have not been described in detail so as not to obscure the present invention.

Although embodiments of the present invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments of the present invention described herein are not constrained to a particular order or sequence. Additionally, some of the described method embodiments of the present invention or elements thereof can occur or be performed at the same point in time.

Orthopedic procedures require some times the use of spine systems, which are in essence fixation assemblies that include intra-pedicular screws and a rod to which the screws are attached. The rod is designed to provide a proper support and position for the fixation assembly.

In accordance with embodiments of the present invention, implantable devices for the spine, for procedures such as spinal fusion surgeries, including (but not limited to) screws such as intra-pedicular screws, hooks, cups, plates, rods and locking devices for rods may be made of composite materials such as carbon polymer composite materials. Such carbon polymer composite materials may include PEEK reinforced typically with about 60% by volume of carbon fibers. For example, such composite materials may include 60-65% by volume of carbon fibers embedded in PEEK. High percentage of carbon fibers in a composite material may provide a composite material having high tensile and stiffness along the longitudinal (fiber) direction. The orientation of the fibers may be controlled to ensure maximal tensile and compressive strength in desired directions. In other embodiments PEEK may be replaced by similar polymers, such as, for example PEKK (Polyetherketoneketone) or PAEK (Polyeryletherketone).

An aspect of the present invention is the provision of a spine system having a novel design and which is made solely or mainly from composite materials such as carbon polymer composite materials. The spine system includes intra-pedicular screws, one or a plurality of connecting rods, and screw connectors for engaging the screws with a rod, to form a fixation device.

Such a spine system may be used in various orthopedic procedures, such as, for example, compression, distraction and reduction.

Reference is now made to the figures.

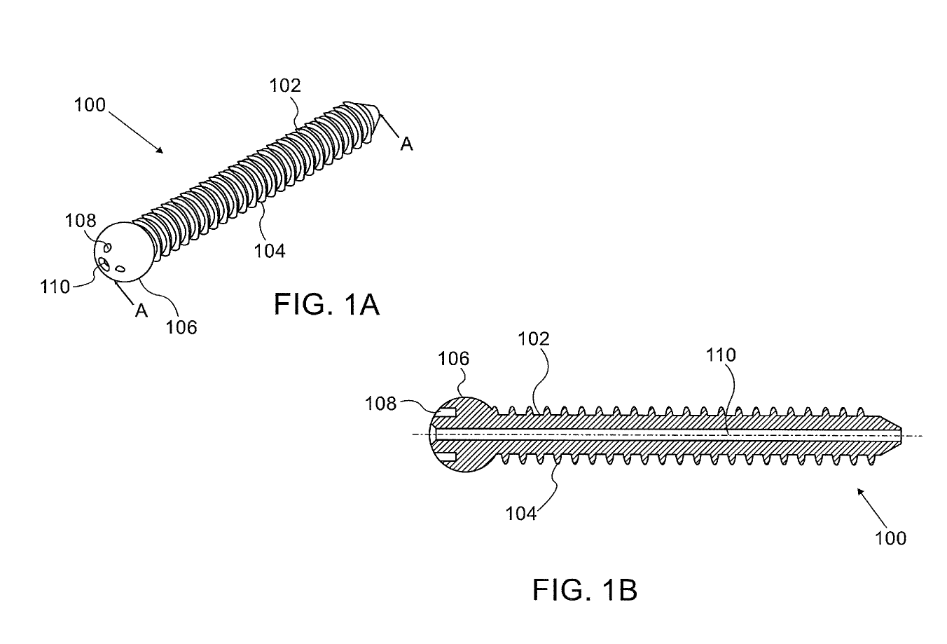

FIG. 1A is an angled view of a screw 100, in particular suitable for serving as an intra-pedicular screw, in accordance with an embodiment of the present invention. FIG. 1B is a cross-sectional view of the screw 100 depicted in FIG. 1A.

Screw 100 may include a screw head 106 and screw body 102, on which a screw thread 104 is provided. The screw may be made, for example, from carbon PEEK, longitudinal carbon fibers, e.g. some 60% by volume of carbon fibers. The screws may be provided in various sizes and diameters, with thread of various pitches and heights, to cater for a variety of needs and situations. The screw head may be designed to interface any specific screw-driver, such as, for example, Allen, Torx, etc. The screw head may include a side hole or a ball-shaped socket.

Duct 110 may be provided within screw 100 across its entire length to allow mounting the screw over a guide wire and advancing the screw to its target during a procedure, e.g., a minimally-invasive procedure. For example, the duct may be designed to accommodate a k-wire having a diameter measuring 1, 1.2, 1.5, or 1.8 mm.

Indentations 108 may be provided on the screw head to accommodate matching tips of a screw-driver, for ease of screwing the screw into its implantation target (e.g. a pedicle of a vertebra).

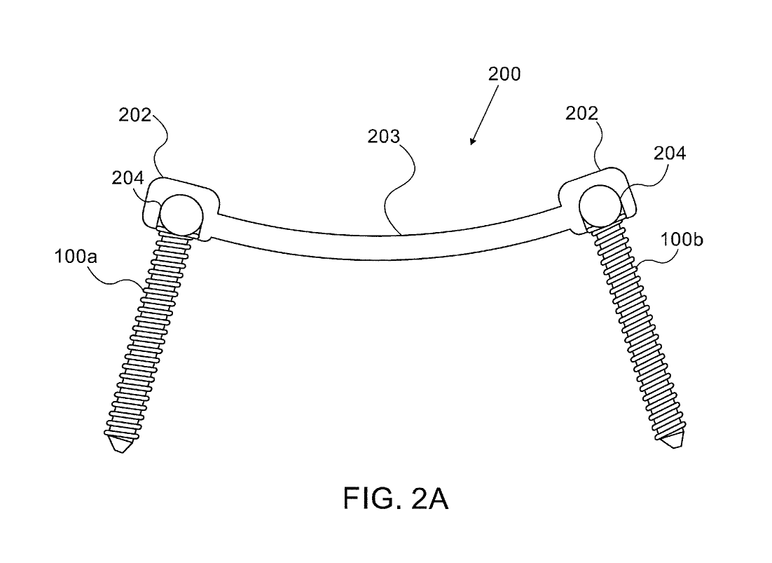

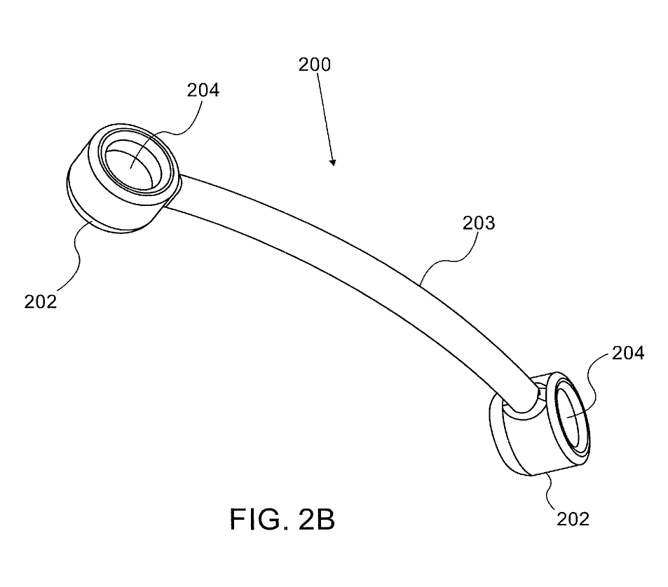

FIG. 2A illustrates a spine system fixation assembly 200 with two end screws, in accordance with an embodiment of the present invention. Spine system fixation assembly 200 may include rod 203, which has a screw connector in the form of receptacle 202 on either ends, defining a socket 204 into which the head of each of the intra-pedicular screws 100a, 100b, may be inserted and fixedly accommodated.

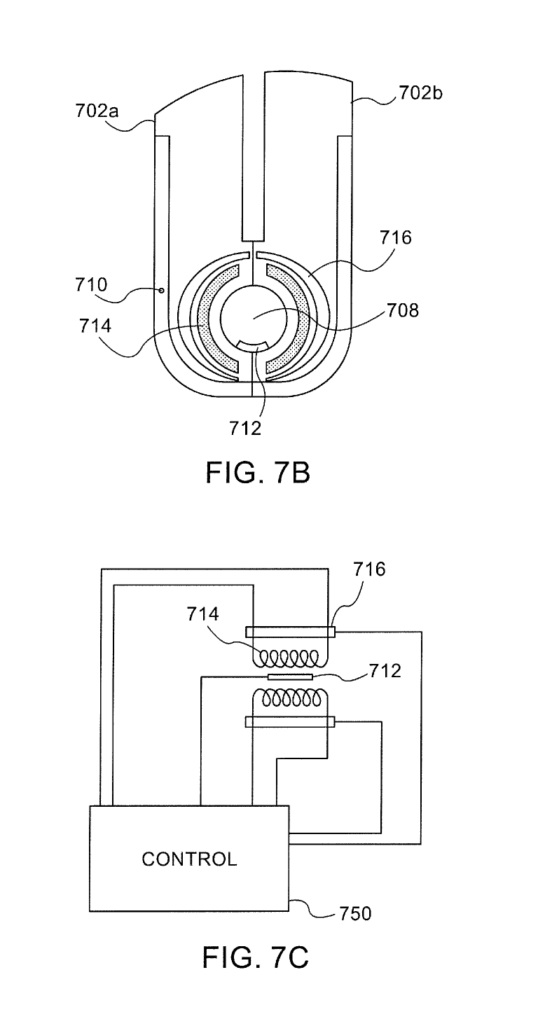

Rod 203 may be provided in a variety of sizes and lengths. In some embodiments of the present invention a spine system kit may be provided which includes a plurality of screws 100a, 100b and a plurality of rods 023 which may be of various lengths sizes and shapes. In some embodiments the shape of the rods may be manipulated (e.g.—see explanation hereinafter with reference to FIGS. 7A-7C).

FIG. 2B illustrates a fixation rod of the fixation assembly 200 shown in FIG. 2A. Socket 204 of receptacle 202 may be designed to receive the head of a screw in a snap-in operation so as to firmly retain the screw head. Other fixing mechanisms may be employed, such as, for example, a retaining lid, a retaining ring, a retaining screw etc.

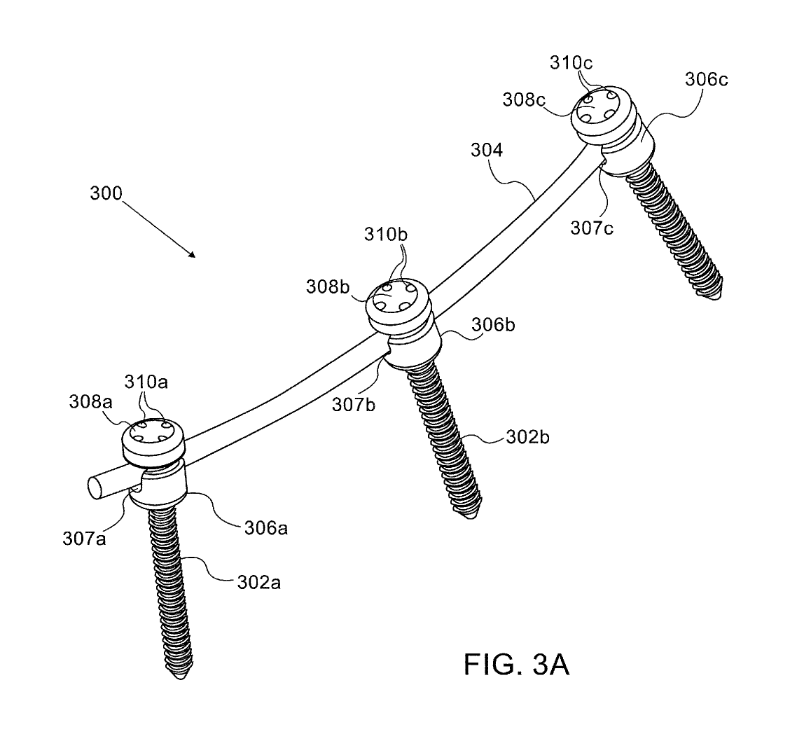

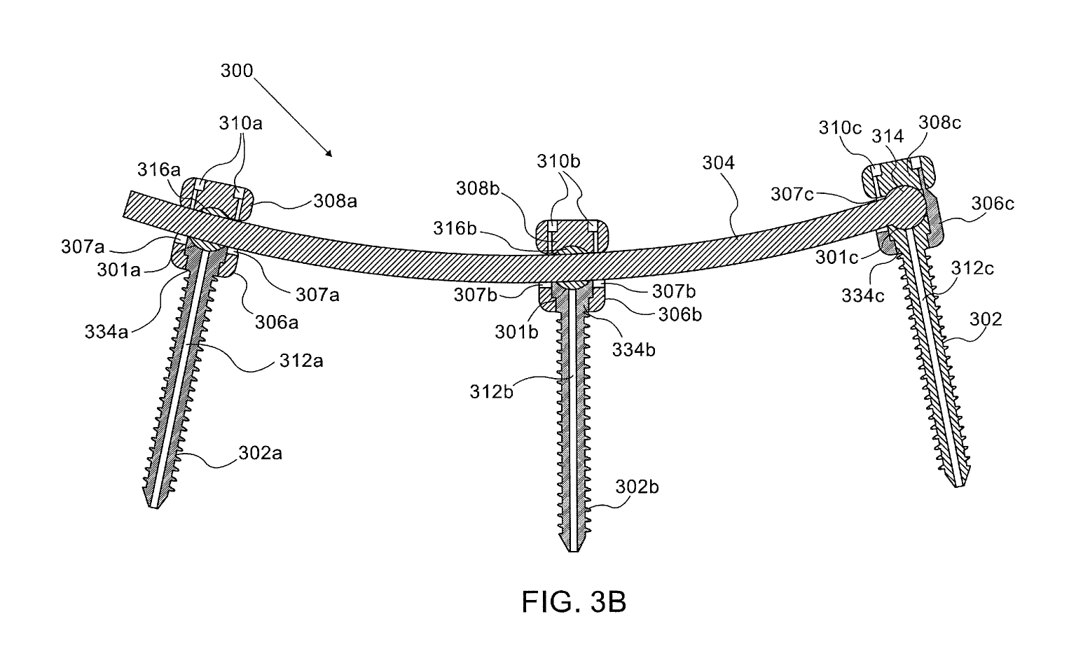

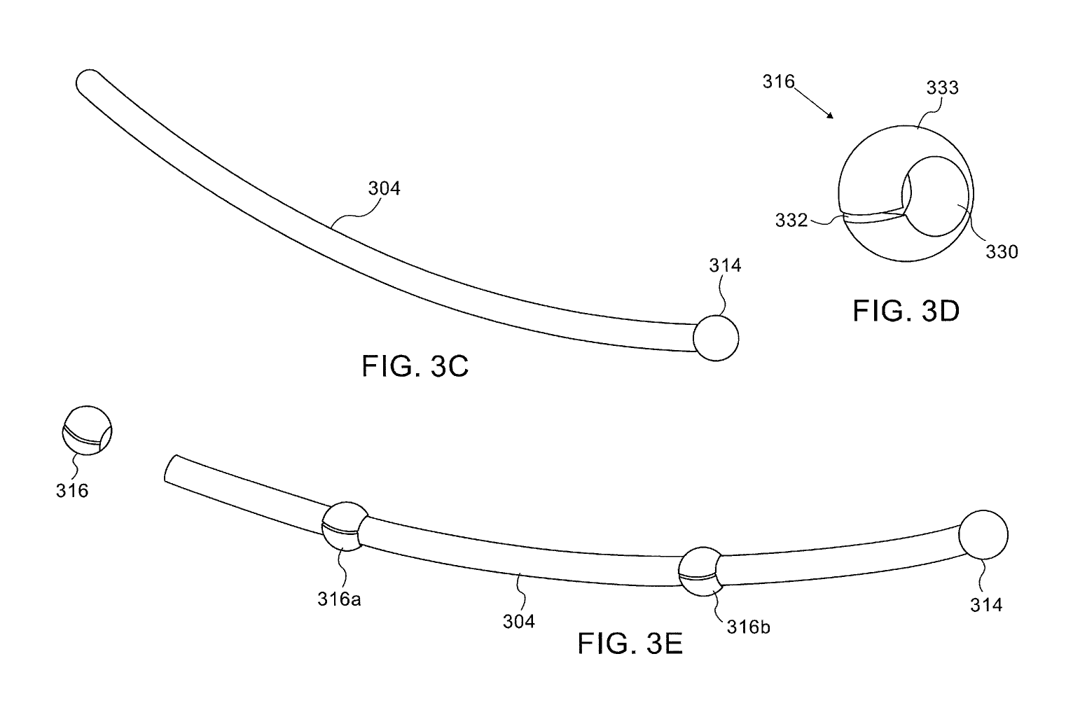

FIG. 3A illustrates a spine system fixation assembly 300 with one end screw 302c and two intermediate screws 302a, 302b, in accordance with an embodiment of the present invention. The screws, 302a, 302b and 302c are all supported and fixed in a predetermined position by rod 304. Screws 302a, 302b and 302c have, each, a screw connector 306a, 306b and 306c respectively, into which the screw is threaded and extends from. Each of the connectors has a bore 307a, 307b and 307c, respectively, extending across the connector, for accommodating rod 304, and a screw top 308a, 308b and 308c, respectively, which is used to engage with that screw connector so as to fasten each of screw connectors to the rod in a desired position along the rod. Designated indentations 310a, 310b and 310c, provided on screw tops 308a, 308b and 308c, respectively, are designed to fit a matching screw-driver to allow convenient screwing of the screw tops onto and off the screw connectors.

FIG. 3B is a cross-sectional view of the spine system fixation assembly 300 shown in FIG. 3A. Each of screws 302a, 302b and 302c, is inserted in and threaded through opening 334a, 334b and 334c of one of the screw connectors 306a, 306b and 306c, respectively, and has on its top a concave contact surface 301a, 301b and 301c, respectively, that matches the convex outer contact surface of fastener bead 316a, 316b and bulbous head 314, respectively. Screw top 308a, 308b and 308c, too, has a concave contact surface that matches the convex outer contact surface of fastener bead 316a, and 316b, and bulbous head 314 located at the end of rod 304 respectively, so that when the screw top 308a, 308b and 308c is screwed on top connector 306a, 306b and 306c respectively, the screw connector is held tight and firmly in position over rod 304. Any of the contact surfaces or all of them may be designed to include some roughness to enhance friction for effective fastening of the screw at the desired location on the rod.

Alternatively, the contact surfaces may include geometrical features to facilitate or enhance locking, such as, for example, dents, grooves, threads and the like.

Some of the elements connecting the screws and the rods may be made from ceramic materials (e.g. zirconium dioxide, known as zirconia).

Each screw may have a duct 312a, 312b and 312c for mounting the screw over a guide wire for guiding the screw to and positioning it at the target implantation location.

FIG. 3C illustrates a connecting rod 304 of the spine system fixation assembly 300 shown in FIG. 3A, with a bulbous head 314 at one end.

FIG. 3D illustrates a fastener bead 316 of the spine system fixation assembly 300 shown in FIG. 3A. Fastener bead 316 has a generally spherical shape with a convex outer surface 333. A bore 330 extends through the bead with a gap 332, defining a slit extending from the outer surface inwardly across the body of the bead to the bore. The slit offers the fastener bead 316 some flexibility allowing easy mounting of the bead over the rod and effectively squeezing it between the body of the screw connector and the screw top and onto the rod (304, see FIG. 3C). FIG. 3E illustrates the connecting rod 304 of the spine system fixation assembly shown in FIG. 3A with bulbous head 314 and fastening bead 316 about to be mounted over rod 304, and fastening beads 316a, 316b already mounted over rod 304. The use of fastener bead 316 offers enhanced stable and sturdy positioning and installment of the spine system.

Rod 400 may be supplied in a variety of lengths, for example having different lengths in steps of 2 mm, for selecting by the surgeon during operation.

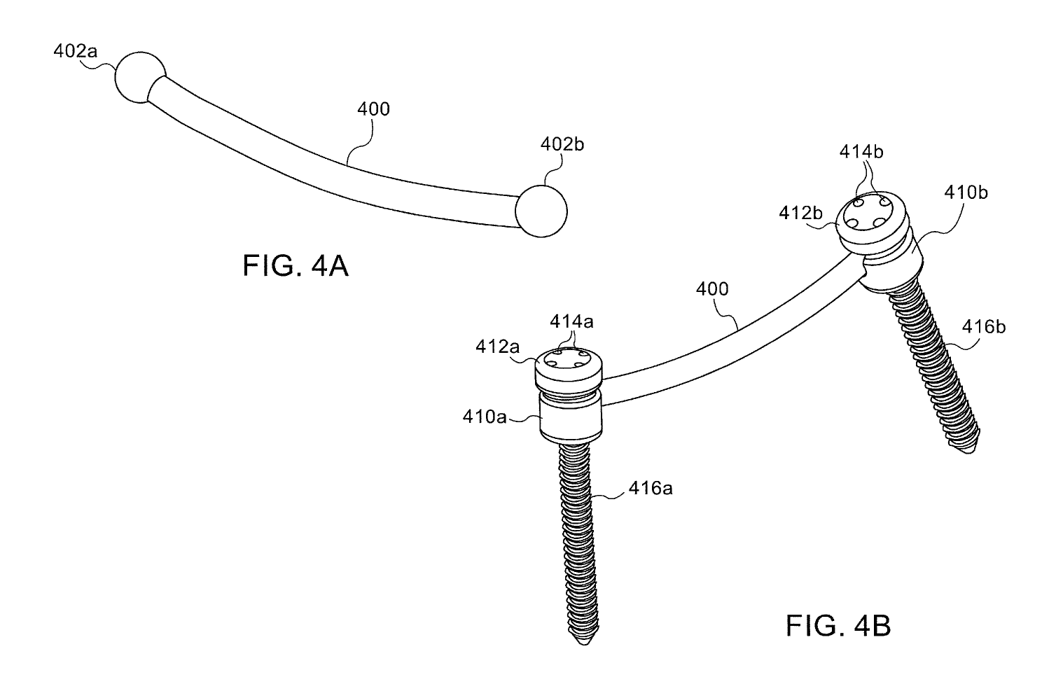

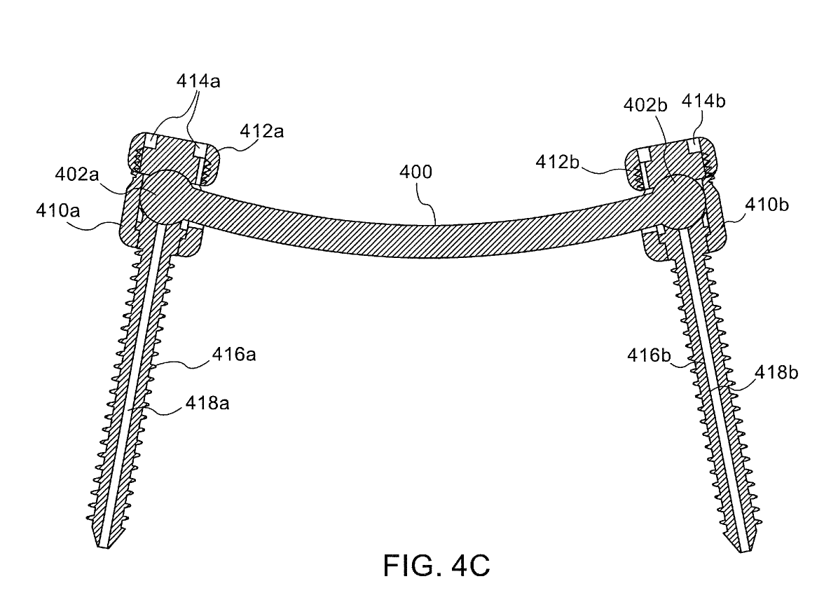

FIG. 4A illustrates a connecting rod 400 for use in a spine system fixation assembly, in accordance with embodiments of the present invention. Rod 400 may have bulbous heads 402a 402b on either ends.

FIG. 4B illustrates a spine system fixation assembly, with two end screws 416a, 416b, in accordance with embodiments of the present invention. FIG. 4C is a cross-sectional view of the spine system fixation assembly shown in FIG. 4B. The screws 416a, 416b may each include a duct 418a and 418b respectively, to allow mounting the screw over a guide wire and guide it to its target implantation location, and are each coupled to a screw connector 410a, 410b respectively, which are positioned at either ends of rod 400 (each holding onto one of the bulbous heads 402a, 402b see FIG. 4C). Screw tops 412a, 412b are screwed on top of screw connectors 410a, 410b respectively, so as to fasten screws 416a, 416b firmly in position onto the bulbous heads of rod 400. Indentations 414a 414b are provide so as to facilitate convenient screwing of screw tops 412a, 412b respectively onto their corresponding screw connectors 410a, 410b, using a matching screw-driver.

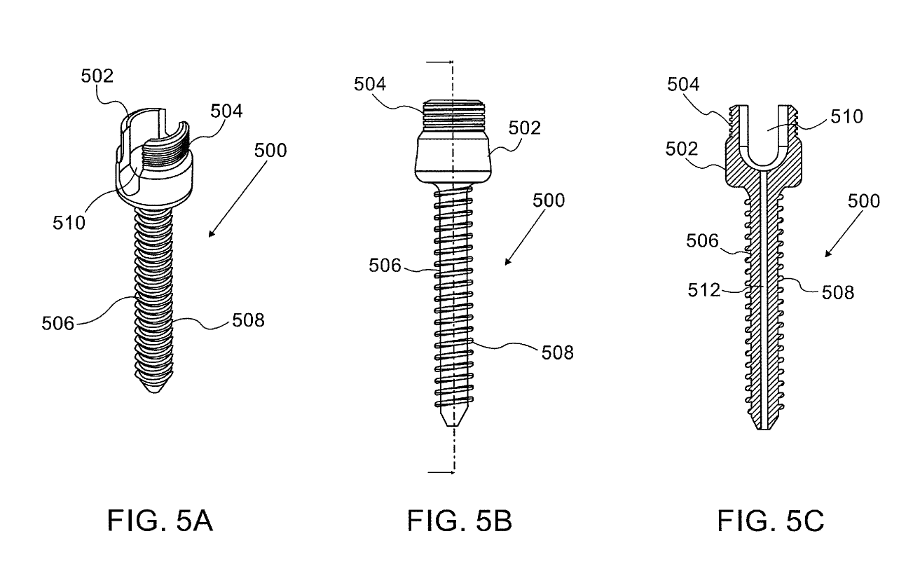

FIG. 5A is an angled view of a screw 500, in particular suitable for serving as an intra-pedicular screw, in accordance with other embodiments of the present invention. FIG. 5B is a lateral view of the screw 500 shown in FIG. 5A, and FIG. 5C is a cross-sectional view of the screw 500 shown in FIG. 5A.

Screw 500 has an elongated screw body 506, with screw thread 508, and may include a duct 512 along the screw body to allow guiding the screw over a guide-wire to a target implantation location.

Screw 500 has an integral screw connector 502 for connecting to a rod of a spine system (e.g., rod 203 of FIG. 2A, rod 304 of FIG. 3A, rod 400 of FIG. 4A). To facilitate this void 510 is provided in screw connector 502 extending laterally across the connector, so as to allow the rod to be inserted in the void 510. A screw top (not shown in this figure, e.g. like screw top 308a, 308b, 308c of FIG. 3A, screw top 412a, 412b of FIG. 4B) may be used to fasten the screw connector 502 to the rod, by screwing it onto the top of screw connector 502, over screw thread 504.

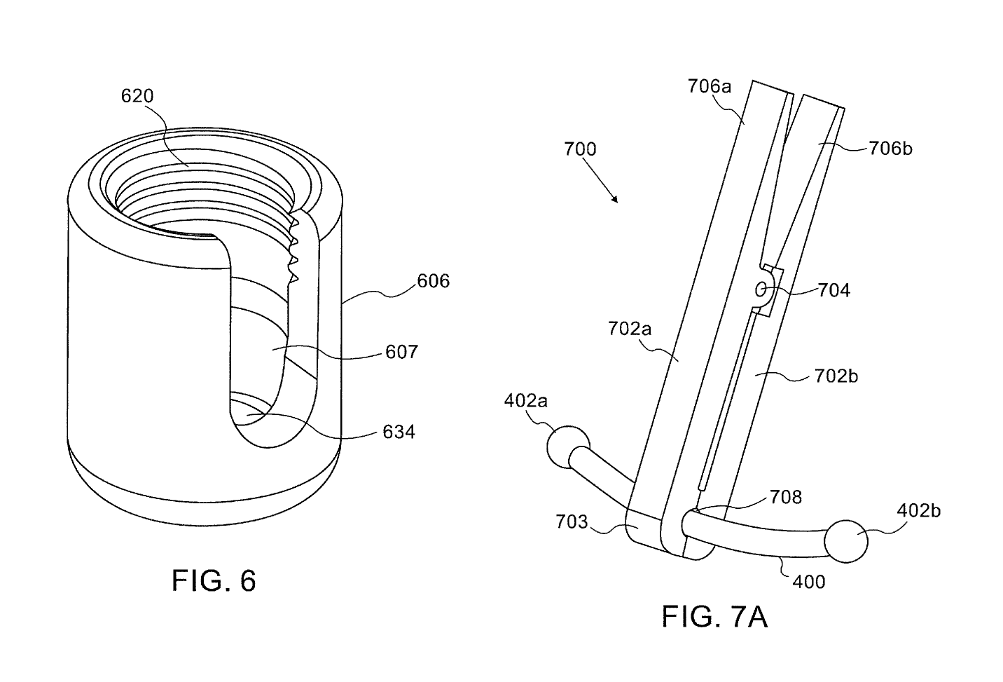

FIG. 6 is an angles view of a screw connector 606, for connecting a screw to a rod of a spine system fixation assembly, in accordance with an embodiment of the present invention. This particular screw connector is designed for connecting a screw to an end of a rod of a spine system, which is why only one opening 607 is provided to inserts the end of the rod into the void within the connector. A bottom opening 634 is provided to allow inserting a screw through that opening leaving the screw head within the screw connector 606. This screw connector has internal threading 620 to allow a screw top (not shown) with external threading to be screwed on top, for fixing the screw connector with the screw onto the rod.

The rod, according to embodiments of the invention, may be made of PEEK, PEEK with chopped carbon fibers, e.g. 10-60% by volume, PEEK with longitudinal carbon fibers, some 60% by volume, or a combination thereof. Metal reinforcement may also be used, to facilitate RF heating or resistance heating so as to allow reshaping of the rod at a desired section or sections of the rod. The rod may include one or a plurality of radiopaque markers, e.g. a tantalum wire along the center of the rod so as to facilitate identifying the rod in medical imaging.

According to embodiments of the present invention, a kit may include a plurality of rods with two bulbous ends (see FIG. 4A), of different lengths for a single-level spine system. According to some embodiments, such a kit may be provided with a plurality of rods with one bulbous end designed to allow mounting of one or a plurality of screw connectors on various positions on the rod other than at its ends, as well as on its ends, and in various rod lengths. In some embodiments such a kit may also include a plurality of screws and screw connectors—separate from or integral to the screws. Furthermore, in some embodiments, such kit may also include fastener beads for enhanced fastening of the screw connectors to the rods.

FIG. 7A illustrates a heating device 700 for heating a rod 400 of a spine system fixation assembly, to allow reshaping the rod, in accordance with an embodiment of the present invention. According to some embodiments of the present invention it is desired to allow the surgeon or the medical technician performing an orthopedic procedure to reshape the rod of the spine system so as to properly fit the rod to the task at hand. In order to do so a heating device 700 is provided.

Heating device 700 may be designed in the form of a clamp having two arms 702a, 702b, which are pressed against each other by means of a spring (not shown) located at pivot 704. At the operating end 703 of the device bore 708 is defined between the arms, designed to receive and hold (e.g. by clamping) a section of the rod and heat it so as to allow reshaping of the rod by bending it at the heated section of the rod. Arms 702a, 702b are provided with tapering ends 706a, 706b respectively across pivot 704 to allow pressing them against each other and releasing the rod from the grasp of the device.

FIG. 7B is a cross-sectional view of the operational end 703 of the heating device shown in FIG. 7A. Heating element 714 is provided about bore 708, designed to heat the section of the rod which is held by the device. The heating element may be, for example, an electric heater, such as a resistor or electromagnetic (RF) heater. Around the heating element 714 a cooling element 716 may be provided, to prevent other parts of the heating device 700 from overheating. The cooling element may dissipate heat by convection, e.g. by employing a cooling system that uses a liquid or gaseous coolant flowing adjacent the heating element, or by conduction, such as a passive cooler, for example, a heat exchanger, or thermoelectric Peltier. In some embodiments element 716 may include insulation to insulate other parts of the heating device 700. External insulation layer 710 may be provided at the operational end 703 of the heating device 700 for additional insulation, so as to prevent infliction of burns to tissue when operating the heating device in-situ. Temperature sensor 712 may also be provided to measure and control the prevailing temperature at bore 708.

FIG. 7C is a schematic illustration of a heating device for heating a rod of a spine system fixation assembly, to allow reshaping the rod, in accordance with an embodiment of the present invention. Controller 750 may be provided to control the operation of heating element 714, and cooling element 716. Controller 750 may use temperature measurements obtained from temperature sensor 712 in an operation algorithm for controlling the heating device.

The heating device may also be designed and used to bend a screw to a desired shape.

Typically, for a rod made solely or mostly of PEEK, the heating device may heat the clamped section to about 250-350 degrees Celsius.

According to embodiments of the present invention, during an orthopedic surgery, the surgeon may determine the length of the rod or rods needed for the patient undergoing that surgery, for example, by using optical markers and a navigational camera, and employ the heating device to bend the rod into the appropriate shape inside the body of the patient or outside the body of the patient, on the operation room table.

While some examples of the present invention were described in detail in the present specification, the scope of the invention is not limited by these examples, and is defined in the appended claims.

1. A spine system comprising:
at least one rod;
at least one screw; and
at least one screw connector configured to connect the at least one screw to the at least one rod,
wherein the at least one rod, the at least one screw and the at least one screw connector are solely or mostly made of a composite material selected from: carbon polymer composite materials, PEEK (polyether ether ketone) reinforced with 60%-65% by volume of carbon fibers embedded therein, and PEEK reinforced with about 60% by volume of carbon fibers embedded therein.

2-4. (canceled)

5. The system of claim 1, wherein at least one of the at least one screw comprises a duct configured to receive a guide-wire arranged to guide that screw to a target implantation location.

6-7. (canceled)

8. The system of claim 1, wherein at least one of the at least one screw connector comprises:
a body having a bore configured to receive at least a portion of the rod inserted therethrough, and
a screw top arranged to engage the screw connector and fasten the screw connector to the rod.

9. The system of claim 1, further comprising at least one fastener bead, over which a respective screw connector is to be fastened to the respective rod.

10. The system of claim 9, wherein the at least one fastener bead has a body with:
a bore passing therethrough configured to enable mounting the bead on the rod, and
a slit cutting therethrough configured to enable flexibly affixing the bead to the respective screw connector, to fasten the screw connector to the rod.

11-12. (canceled)

13. The spine system of claim 1, configured as a kit comprising:
a plurality of rods having a variety of lengths for selecting by a surgeon during operation;
a plurality of screws; and
a plurality of screw connectors.

14-20. (canceled)

21. The system of claim 1, wherein the at least one screw comprises respective indented screw heads.

22. The system of claim 10, wherein the at least one fastener bead is slidably mountable on the at least one rod.

23. The system of claim 1, wherein the at least one screw connector is configured to receive the at least one screw, the system further comprising at least one locking element configured to connect and lock the at least one screw connector to the at least one rod.

24. A method of configuring a spine system, the spine system comprising at least one rod and respective sets of screws, screw connectors and fastener beads, wherein the at least one rod, the sets of screws and the sets of screw connectors are solely or mostly made of a composite material selected from: carbon polymer composite materials, PEEK (polyether ether ketone) reinforced with 60%-65% by volume of carbon fibers embedded therein, and PEEK reinforced with about 60% by volume of carbon fibers embedded therein, the method comprising:
connecting the screw connectors to respective screws,
mounting the fastener beads onto the rod, and
affixing, flexibly, the fastener beads to respective screw connectors to fasten the screw connectors to the rod.

25. The method of claim 24, further comprising mounting the fastener beads slidably onto the at least one rod prior to the affixing.

26. The method of claim 24, further comprising guiding at least one of the screws to a target implantation location using a guide-wire passing therethrough.

27. The method of claim 24, further comprising selecting one of a plurality of rods having a variety of lengths.

28. A method comprising:
implanting a plurality of screws into target implantation locations,
connecting a plurality of respective screw connectors to the plurality of screws,
mounting a plurality of fastener beads onto at least one rod according to respective target implantation locations, and affixing, flexibly, the plurality of fastener beads to the plurality of respective screw connectors to fasten the plurality of screw connectors to the rod,
wherein the at least one rod, the plurality of screws and the plurality of screw connectors are solely or mostly made of a composite material selected from: carbon polymer composite materials, PEEK (polyether ether ketone) reinforced with 60%-65% by volume of carbon fibers embedded therein, and PEEK reinforced with about 60% by volume of carbon fibers embedded therein.

29. The method of claim 28, further comprising selecting, by a surgeon during operation, at least one of a plurality of rods having a variety of lengths as the at least one rod.

30. The method of claim 28, wherein implanting at least one of the screws is carried out by guiding the screw to the respective target implantation location using a guide-wire passing through the screw.

31. The method of claim 28, wherein implanting at least one of the screws is carried out via a respective indented screw head.

32. The method of claim 28, further comprising mounting the fastener beads slidably onto the at least one rod prior to the affixing.
